(19) United States
(12) Patent Application Publication
(10) Pub. No.: US 2008/0051659 A1
(43) Pub. Date: Feb. 28, 2008

(54) Ultrasonic Diagnostic Apparatus

(76) Inventors: Koji Waki, Chiba (JP); Naoyuki Murayama, Chiba (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/629,918

(22) PCT Filed: Jun. 16, 2005
(86) PCT No.: PCT/JP05/11032

§ 371 Date: Dec. 18, 2006

(30) Foreign Application Priority Data
Jun. 18, 2004 (JP) 2004-180-830

(51) Int. Cl. A61B 8/00

(52) U.S. Cl. 600/443

(57) ABSTRACT

An ultrasonic diagnostic apparatus (ultrasonic elastograph) for measuring strain or elastic modulus of a tissue or an organ of a living body, creating a strain image or an elastic modulus image by assigning a hue to the measurement data corresponding to the measured value, combining these images with a black and white tomogram, displaying the synthetic image on a color monitor, and displaying the hue assigned to the measurement data with a color bar. Information on comparison between a measured value corresponding to the hue of the color bar displayed on the color monitor and the reference value of the measured value is displayed at a position adjacent to the color bar on the screen of the color monitor.

### TECHNICAL FIELD

The present invention relates to an ultrasonic diagnostic apparatus, in particular, to the ultrasonic diagnostic apparatus for creating a strain image of an organ in a living body that contributes to medical diagnosis.

### BACKGROUND ART

The ultrasonic diagnostic apparatuses transmit ultrasonic waves to the inside of the body of an object to be examined, detect echoes reflected from the body tissues, create and display images of those reflected signals. Images displayed in these apparatuses are tomograms presenting tissue characterization of the body of the object which is measured approximately in real time by applying an ultrasound probe on the surface of the object, or images presenting blood flow or movement of organs being measured with application of Doppler effect.

Upon displaying images of blood flow or movement of organs using an ultrasonic diagnostic apparatus, blood flow or movement of organs is color displayed on the monitor with black and white tomogram as a background image. In this type of display method, it is common to assign different hues to the measurement data of blood flow or movement of organs according to its movement speed, and to display a color bar of the assigned hue on the corner of a monitor screen.

Recently the development of an advanced technique of ultrasonic diagnostic apparatus has been promoted, which is for obtaining the correlation between the images measured in different times, measuring the amount of displacement of a body tissue during that time, for example, the amount of strain from displacement of the tissue, or measuring elastic modulus of the tissue by artificially giving pressure change from outside, and for imaging and displaying them on a monitor (See Patent Documents 1 and 2).

An imaging technique for displaying images of the measurement result of strain magnitude or elastic module of body tissues measured by ultrasonic waves is defined here as an ultrasound elastography, and hue information such as red, blue and others are assigned also to measurement data upon displaying these images according to the measured amount of strain or elastic module. Especially to scleritic portions such as cancer or tumor, the hue information distinguishable from other tissues are assigned and displayed on a monitor. Such technique is disclosed in, for example, Patent Document 3.

In the meantime, the hardness of tissues of cancer or tumor in the body varies depending on the region, personal differences, or condition of diseases. Consequently there is a problem in that it is difficult for a doctor to identify the measurement data of strain magnitude or elastic module given with, for example, hue information of which the three primary colors in light formed by R(red), G(green) and B(blue) is displayed in a rectilinear gradation that changes from red to blue. As an answer to this problem, a technique for varying the hue for assigning to measurement data in a staircase pattern is provided (see Patent Document 4).

Patent Document 1: U.S. Pat. No. 5,107,837

Patent Document 2: JP-1993-313713A

Patent Document 3: JP-2000-60853A

Patent Document 4: WO 2005/048847A

Though the display technique for assigning the hue to the ultrasonic measurement data is described above taking ultrasound elastography as an example, the color bar indicates only the range of measurement data and it has been difficult to grasp the measurement data quantitatively.

### DISCLOSURE OF THE INVENTION

Problems to be Solved

In the above-mentioned conventional ultrasound elastography, since the diseased area that is harder than the surrounding tissues can be displayed in a distinguishable manner on a monitor along with a color bar, it is possible for a doctor to identify that the area is harder than the surrounding tissues. However, degree of hardness of the displayed tissues comparing to the surrounding tissues could not be acknowledged quantitatively.

Also, even though a doctor can specify the hue of the target region in the strain image to which the hue information is assigned since the hue of color bars are displayed with gradation, they have to spend much time for specifying where the hue falls in the color bar which lowers the efficiency of diagnosis.

The objective of the present invention is to provide an ultrasonic diagnostic apparatus that makes it possible for a doctor to grasp hardness of the affected area by being able to observe the strain image or elastic module image more quantitatively compared to the prior art, and improves efficiency of diagnosis thereof.

Means to Solve the Problems

In order to achieve the above-mentioned objective, an ultrasonic diagnosis apparatus of the present invention creates color images of the strain of the tissues measured in a living body by ultrasonic waves according to the amount of strain and displays them on the color monitor along with a color bar of the assigned hue information, wherein the ultrasonic diagnostic apparatus is characterized in comprising means to display at least one of the hue information corresponding to the average value or maximum value of the measured strain adjacent to the color bar.

Also, in order to achieve the above-mentioned objective, the present invention is characterized in adding means to the ultrasonic diagnosis apparatus for specifying the hue, when specific positional information is inputted from the strain image displayed on a color monitor, corresponding to the position on the color bar and displaying the information of comparison corresponding to the average value or maximum value of the strain of the specified phase information.

### BRIEF DESCRIPTION OF THE DIAGRAMS

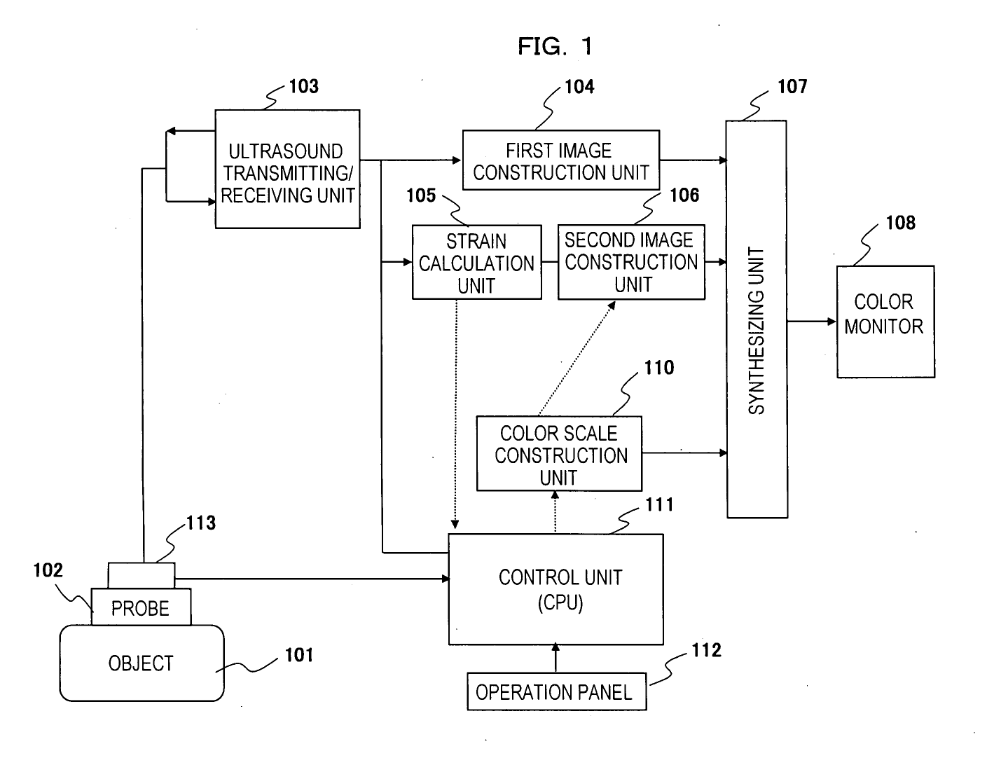

FIG. 1 is a block diagram showing the general configuration of an ultrasonic diagnostic apparatus of one embodiment in the present invention.

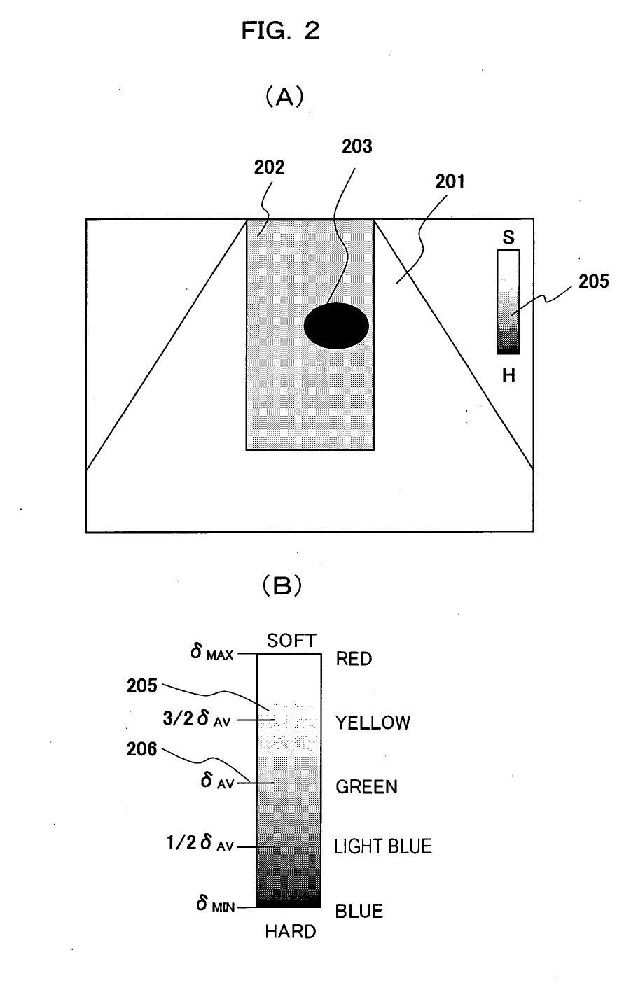

FIG. 2 is a diagram showing of a first image display pattern and details of a color bar of in present invention.

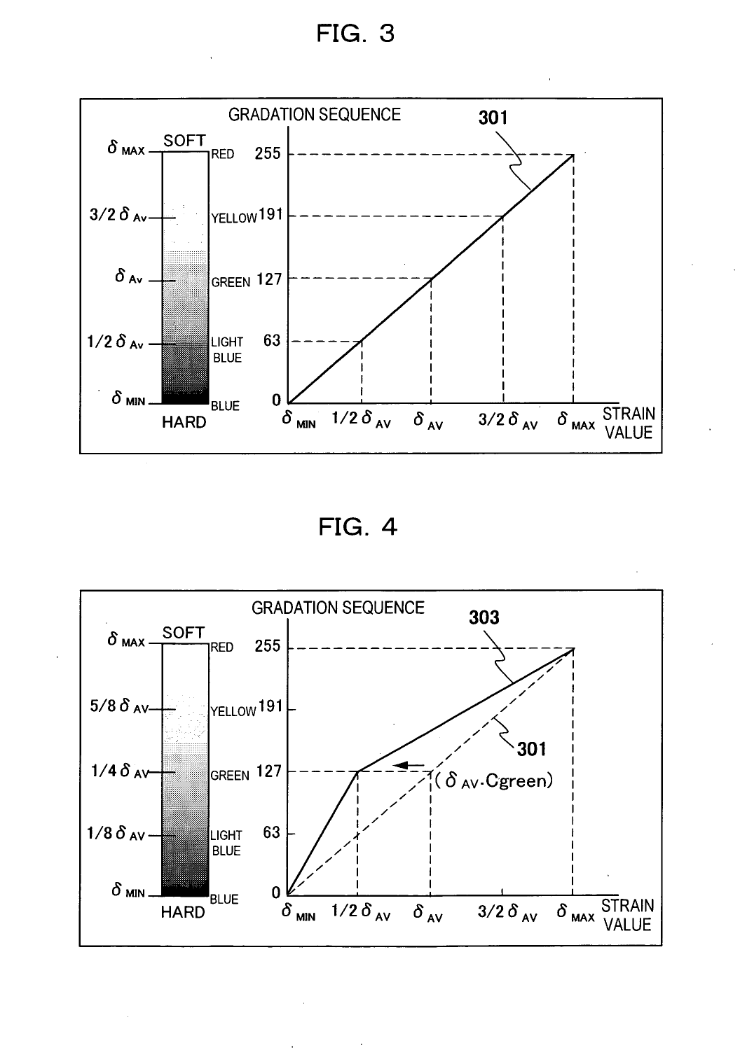

FIG. 3 is the first embodiment in the present invention showing the relationship between the strain and hue information.

FIG. 4 is a second embodiment in the present invention showing the relationship between the strain and hue information, and a method thereof.

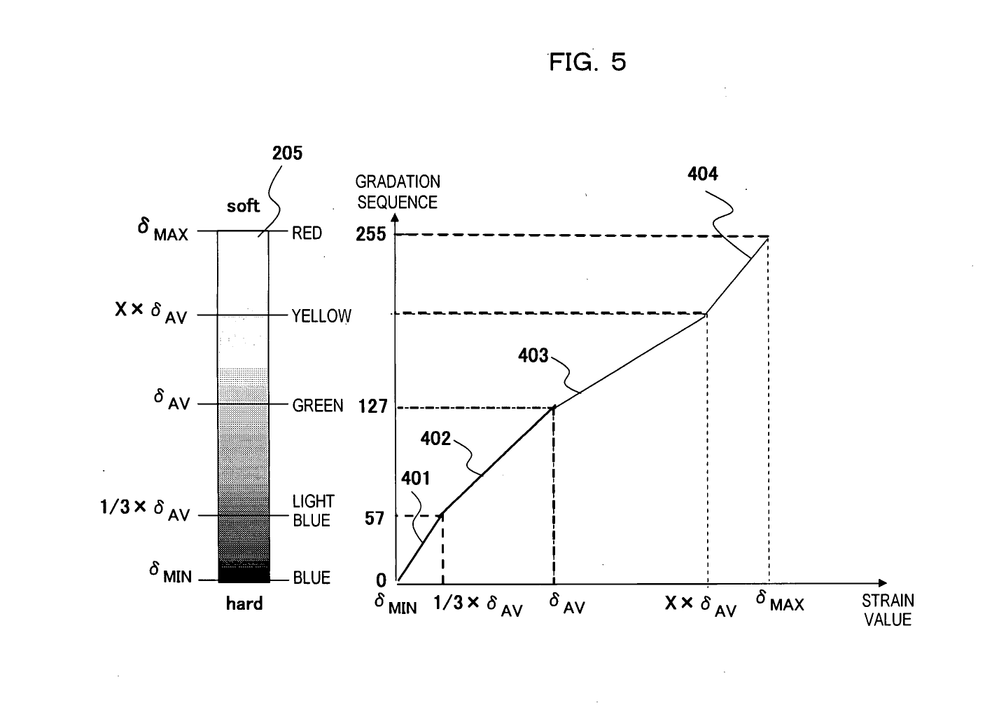

FIG. 5 is a third embodiment of the present invention showing the relationship between the strain and hue information.

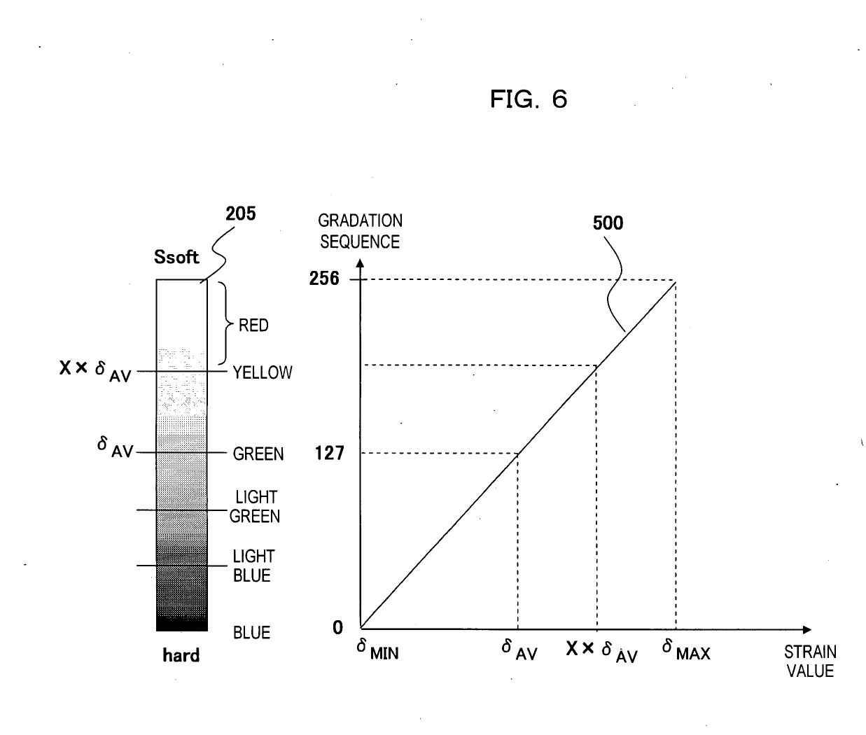

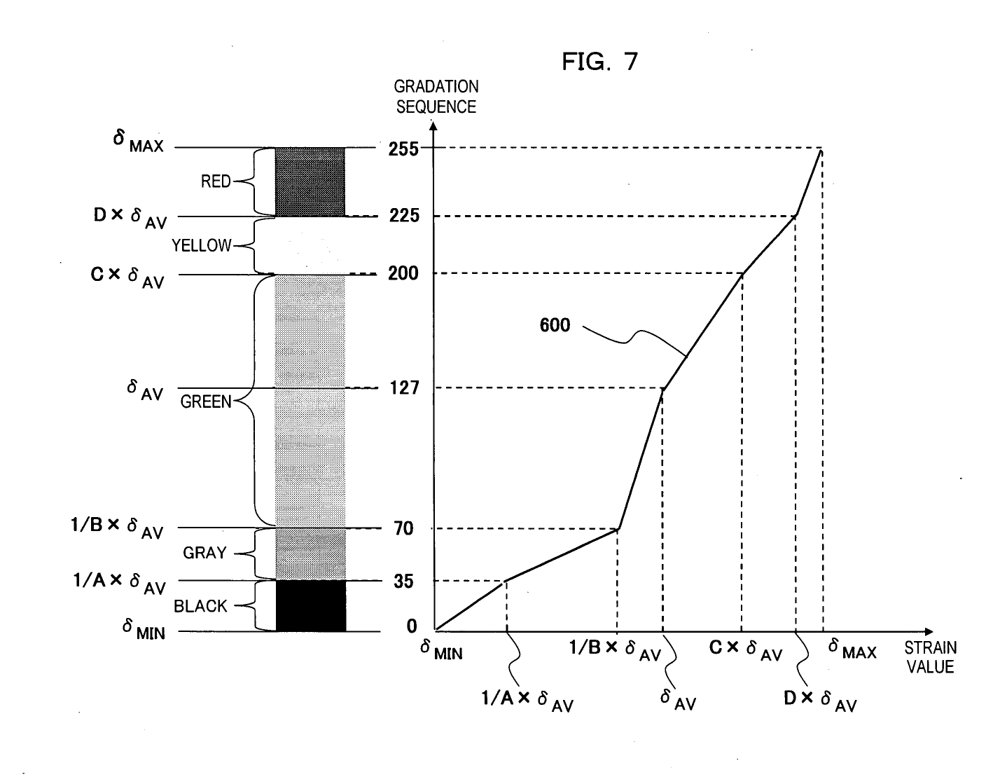

FIG. 6 is a fourth embodiment of the present invention showing the relationship between the strain and hue information.

FIG. 7 is a fifth embodiment of the present invention showing the relationship between the strain and hue information.

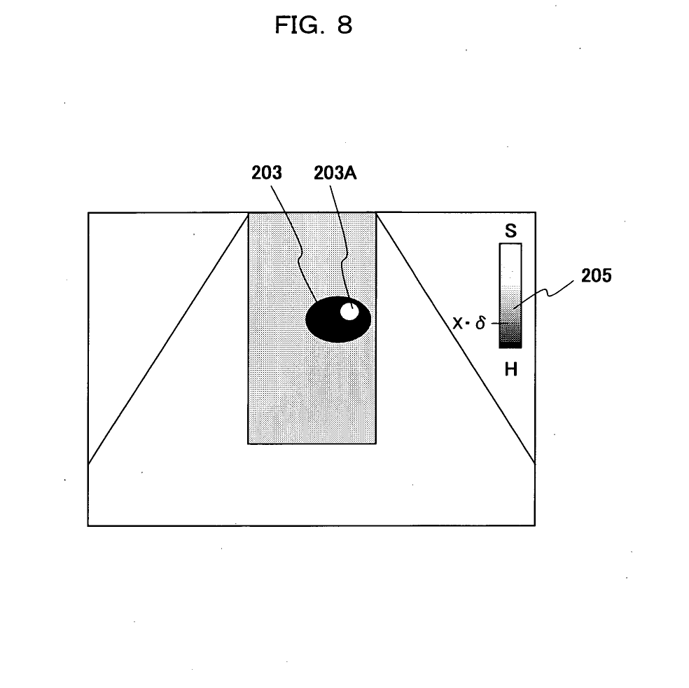

FIG. 8 is a diagram showing an embodiment for specifying the hue from positional information on a screen.

### BEST MODE FOR CARRYING OUT THE INVENTION

Hereinafter, embodiments of the present invention will be described referring to the diagrams. As seen in FIG. 1, an ultrasonic diagnostic apparatus to which the present invention is applied comprises:

ultrasound probe 102 for applying to object 101, and transmitting ultrasonic beams to object 101 as well as receiving ultrasonic waves reflected in the body of object 101;

a transmitting circuit for providing transmitting signals that transmit ultrasonic waves to object 101 with a predetermined time interval;

a receiving circuit for receiving echoes reflected in the body of object 101, converting them into electronic signals (echo signals) and outputting them;

ultrasound transmitting/receiving unit 103 provided with a phasing addition circuit for forming ultrasonic beam signals (RF signal data) by executing phasing addition process on the echo signals being outputted from the receiving circuit, and outputting them;

first image construction unit 104 for constructing a tomogram, for example, a black and white tomogram of the cross section to which ultrasound probe 102 is applied on object 101 using RF signal data being outputted from the phasing addition circuit;

strain calculation unit 105 for calculating the strain data (may also be described as “elasticity data”) by measuring displacement of the tissues of object 101 from the RF signal data;

second image construction unit 106 for constructing colored strain images or colored elastic images based on the strain data or elasticity data;

image synthesizing unit 107 for creating a single image by synthesizing the black and white tomogram and images such as the strain image;

color monitor 108 for displaying the synthesized images;

color scale constructing unit 110 for creating a color scale (color bar) to be displayed on color monitor 108;

control unit (CPU) 111 for controlling the previously mentioned components; and

operation panel 112 provided with a key board, operation key, mouse, joystick or trackball for inputting the respective orders to CPU 111.

Next, acquisition of ultrasound strain images and operation of the display thereof will be described. When a mode switch for acquiring strain images (not shown in the diagram) provided in operation panel 112 is manipulated by an operator, transmitting signals from the transmitting circuit are provided to a plurality of transducer elements being arrayed in ultrasound probe 102. The transducer elements are activated by this transmission, and ultrasonic waves focused to the depth point (focus point) appointed beforehand in a predetermined direction inside of object 101 are outputted. Then ultrasound probe 102 receives the echoes reflected inside of object 101. The echoes received by ultrasound probe 102 are made into electrical echo signals in the receiving circuit. After these echo signals are amplified, dynamic focusing process is implemented on the signals in the phasing addition circuit in relation to the transmission direction. Ultrasonic beam signals are formed by the implementation of this process. These ultrasonic beams receive the processes such as gain-compensation, logarithmic compression, demodulation, edge enhancement and dynamic filtering in the respective sections such as gain compensation section, logarithmic compression section, demodulation section, edge enhancement section and filtering section. After receiving such processes, the signals are inputted to first image construction unit 104 and also to strain calculation unit 105.

The above-mentioned ultrasound transmitting/receiving operation is carried out from one end to the other end of the predetermined ultrasound measuring scope changing directions under the control of CPU 111. By such scanning of ultrasonic waves the image data of the cross section in a body of an object to which ultrasound probe 102 is applied is obtained, the obtained image data is written in to the storage media which is generally called black and white scanning converter, for example, the frame memory or cine memory in first image construction unit 104, and a tomogram is thus constructed. Then the ultrasound scanning is repeatedly executed at a predetermined time interval (frame rate), and a plurality of images is recorded to the frame memory or cine memory by ultrasonic beam signals being obtained every transmitting/receiving cycle of the ultrasonic waves in increments of frames. The image data recorded in the media such as frame memory is sequentially read out in a timing of synchronized signals of color monitor 108 which are irrelative to the transmission/reception of ultrasonic waves, for example, a timing of horizontal synchronized signals, scan-converted and displayed as a black and white tomogram on a screen of color monitor 108.

The ultrasonic beam signals obtained by the above-mentioned ultrasound transmission/reception or ultrasound scanning are inputted to strain calculation unit 105, and the strain calculation to be described below is carried out. The storage media for storing the inputted ultrasonic beam signals in increments of the frames is provided also in strain calculation unit 105, and the ultrasonic beam signals for one frame of the Nth (N=sequentially updated integer as 1, 2, 3 . . . ) frame of the adjacent ultrasound scan is stored in this storage media.

Upon obtaining the ultrasonic beam signals by the (N+n)th (“n” is an arbitrary integer) scan, CPU 111 executes the correlation processing between the ultrasonic beam signals of the Nth scan and the ultrasonic beam signals of the (N+n)th scan in relation to strain calculation unit 105. By such process, the displacement or displacement vector (the direction and size of displacement) of the respective measurement points on the tomogram between those scans is calculated, and the displacement image data is created. As for the correlation processing, either the method for applying to the respective ultrasonic beam signals of the plurality of ultrasonic beams which constructs the frame data or the method for applying two-dimensional correlation between the frame data of the Nth scan and the frame data of the (N+n)th scan may be used to implement one-dimensional correlation between the ultrasonic beam signals in the same direction of the Nth scan and the (N+n) th scan.

As for the two-dimensional correlation method, well-known methods such as the block matching method or the gradient method can be used. The block matching method is a method to segmentize images into a plurality of blocks setting, for example, M×M pixels as one block, and to search for an imaging block obtained by the Nth scanning which is the most approximated to a focused block in an image being obtained by the (N+n)th scanning. By doing so the detection on how much and in what direction the displacement is made along with passage of time between those blocks is implemented. Through carrying out this detection a plurality of times by changing the focused blocks, the displacement data in increments of the blocks can be obtained. Using this displacement data in increments of the blocks, estimated calculation of the displacement of the respective pixels that are constructing an image is performed. By this calculation, the displacement data distribution of the respective pixels can be obtained. And the strain image data can be acquired by performing spatial differentiation on this displacement data distribution in strain calculation unit 105.

The acquired strain image data is sent to image synthesizing unit 107. The tomogram data obtained in the (N+n)th scanning is also provided to image synthesizing unit 107, and in image synthesizing unit 107 the tomoram data obtained in the (N+n)th scanning and the strain image data calculated between the measurement data of the Nth scanning and the (N+n)th scanning are synthesized by matching the image address of the respective image data. Since the objective of this image synthesizing is to observe the strain condition of the organ or tissue of a living body, it is preferable to display the strain image by assigning the hue formed with R(red)˜G(green)˜B(blue) so that the strain image data of the focused organ or tissue can be identified by an observer more easily from the tomogram data.

For this purpose, second image construction unit 106 comprises a gradation sequence unit for gradating the inputted signals, and a color scan converter for storing the image data and reading out the stored image data for the purpose of display corresponding to the display synchronized signals of color monitor 108. More specifically, the strain image data outputted from strain calculation unit 105 is made into signals of 8-bit configuration (256 gradations) in the gradation sequence unit in order to be allocated to the gradation sequence of 256 gradations, and they are outputted to the color scan converter. The color scan converter comprises the color encoder and frame memory, and the hue of R(red)˜G(green)˜B(blue) corresponding to the relationship between the gradient sequence and the hue which is set in advance is assigned to the 8-bit strain image data being outputted from the gradation sequence unit and inputted to color encoder, and written in to the frame memory. Then CPU 111 coordinates the address of the content of the frame memory in the black and white scan converter and the content of the frame memory in the color scan converter, reads them out, and outputs them to image synthesizing unit 107. As a result, the color strain image and the black and white strain image are synthesized and displayed on the screen of color monitor 108.

As for the synthesizing method of the strain image data to which the hue is assigned and the black and white tomogram data, a variety of methods can be cited. For example, as for the pixel address having both strain image data and tomogram data there are methods such as: (i) a method to select strain image data by priority as image data; and (ii) a method to add tomogram data and strain image data by the predetermined ratio, and either method may be used.

Also, color bar 205 indicating the relationship of which the gradation sequence is converted into hues is displayed on a display screen of color monitor 108. An example of this color bar is shown in FIG. 2, and it changes the color sequentially from Red˜Yellow˜Green˜Light-blue˜Blue with a gradation from the upper part to the lower part of the screen. As for the relationship between the strain image data and the hue, the red-color code is allotted to the portion where a large strain is measured (soft portion) and the blue-color code is allotted to the portion where a small strain is measured (hard portion). To the portion where the strain is measured approximately in average value (δ_{AV}), the green-color code is allotted. Also, in case the strain is more than the average value (δ×X) yellow which is the color between red and green is allotted, and light blue which is between green and blue is allotted in case the strain is less than the average value (δ_{AV}X 1/Y). In accordance with the above-mentioned allotment of the strain and hue, colors are assigned to the strain image. In addition, on the upper end that is the red-color side of color bar 205, the term “soft” is displayed to indicate that the red color means the tissue is soft, and the term “hard” is displayed on the bottom end that is the blue-color side to indicate that the tissue is hard.

The above-mentioned color bar 205 is composed of color scale construction unit 110. More specifically, color scale construction unit 110 is provided with the display memory that is not shown in the diagram in order to display color bar 205 to the screen of color display monitor 108. Color bar 205 is displayed by writing in the data for displaying the color bar to the predetermined address region deviated from the ultrasound image display region of this display memory. The display memory may be provided specifically for displaying color bar 205, or it may be shared with the character memory or graphic memory for displaying ID of an object.

Next, different display modes of the relationship between the hue of the color bar and strain value will be described. As shown in FIG. 2, ultrasound tomogram 201 is displayed along with the strain image including said ultrasound tomogram 201 and diseased portion 203 being superimposed. Here the strain image is measured with regard to region of interest (ROI) 202 being set on the tomogram in advance. The reason for this is because there is not much worth in obtaining the calculation of the strain for the entire scope of the ultrasound measurement. More specifically, since the portion that an examiner is interested in is the strain with regard to a specific part of the entire scope of the ultrasound measurement, the calculation of the strain over the entire scope would not be of much worth to the examiner. Furthermore, performing the calculation of the strain for the entire scope of the ultrasound measurement takes extra time before displaying the strain image that leads to the lowering of the frame rate of display or examination efficiency. Moreover, since the pressure applied from the body surface in the area deeper than a certain depth of the object does not operate in a predetermined direction but disperses, it is difficult to measure the strain accurately and the noise would also be increased. In addition, the above-mentioned ROI 202 is set by the examiner through manipulating the ROI inputting device such as trackball or mouse provided in keyboard 112.

First, the standard display mode of the relationship between the strain value measured in the above-mentioned ROI 202 and the hue of color bar 205 will be described using FIG. 3. CPU 111 calculates the distribution of the strain value in ROI 202 using the previously mentioned method. Then CPU 111 performs addition of the strain value in ROI 202, and calculates the total amount of the strain value in ROI 202. The total amount of the strain value is divided by the pixel value in ROI 202, and the average value of the strain value in ROI 202 (δ_{AV}) is calculated. This average value of the strain value (δ_{AV}) is allocated to green color which is the center of the red˜green˜blue color bar. Next, the maximum value of the strain value (δ_{MAX}) is allocated to red color and the minimum value of the strain value (δ_{MIN}) is allocated to blue color. In other words, this first display mode gives the hue to the strain value from its minimum value (δ_{MIN}) to the maximum value (δ_{MAX}) in ROI 202 with its average value as median so that the hue changes from blue to red linearly. In addition, since it is difficult to grasp the relative comparison of the strain amount between the respective hues if only color bar 205 is displayed adjacent to an image, the comparison value with regard to the representative hues of color bar 205, for example, three hues of green color indicating the average value of the strain value (δ_{AV}), yellow color indicating intermediate value between the maximum value and the average value of the strain value (3/2 δ_{AV} or 3/4 δ_{MAX}) or light blue color indicating intermediate color between the minimum value and the average value of the strain value (1/2 δ_{AV} or 1/4 δ_{MAX}) is displayed. As for the comparison value the ratio of intermediate value between the maximum value and the average value of the strain value indicated in yellow and intermediate value between the minimum value and the average value of the strain value indicated in light blue in relation to the average value of the strain value (δ_{AV}) is displayed in the numeric value or mark as shown in FIG. 3.

In case of displaying the above ratio by numeric values, values between the maximum value (δ_{MAX}) and the minimum value (δ_{MIN}) of the strain value being measured in strain calculation unit 105 are allocated to 256 (0˜255 or 1˜256) gradations under control of CPU 111, the maximum value thereof δ_{MAX}) is set as 256 (or 255), the minimum value (δ_{MIN}) as 1 (or 0) and the average value (δ_{AV}) as 128 (or 127), and intermediate value between the maximum value and the average value (3/2 δ_{AV} or 3/4 δ_{MAX}) is further set as 192 (or 191), intermediate value between the average value and the minimum value (1/2 δ_{AV} or 1/4 δ_{MAX}) as 64 (or 63). Then these numeric values are displayed at the point applied to the position associated by color bar 205 or the position adjacent to stick-like mark 206. It is preferable that the marks are applied so that the color and the mark are correspondent to each other with the purpose not to erase the color inside of color bar 205.

Furthermore, in case of displaying the above mentioned ratio by magnifying power, the values of the maximum value (δ_{MAX}), minimum value (δ_{MIN}), intermediate value between the maximum value and the average value (3/4 δ_{MAX}) and intermediate value between the minimum value and the average value (1/4 δ_{MAX}) divided by the average value (δ_{AV}) are displayed. For example, if this calculation is performed on the above-mentioned relationship, the maximum value (δ_{MAX}turns out as 2 δ_{MAX}, minimum value (δ_{MIN}) turns out as 0, intermediate value between the maximum value and the average value as 1.5 δ_{AV} and intermediate value between the minimum value and the average value as 0.5 δ_{AV}.

In this manner, by applying the numeric value or mark for representing relative ratio magnifying power of strain to color bar 205, an examiner can grasp the hardness or softness of the region where ROI is most prone to strain or the region having the average strain in the strain image.

While the above-mentioned display mode of color bar is an example of converting the values between the maximum value and the minimum value of strain into the hue linearly, the present invention is not limited to this mode. The present invention includes the mode to convert the values between the maximum value and the minimum value of strain into the hue nonlinearly. Next, the display mode thereof (the second display mode) will be described.

The second display mode of the present invention is illustrated in FIG. 4. This second display mode is suitable for extracting the strain variation of the relatively hard portion (region with small strain) in the body in minute detail. More specifically, while it is the same as the first display mode in that the measured maximum value of the strain (δ_{MAX}) is allocated to red color and the minimum value of strain (δ_{MIN}) is allocated to blue as shown in FIG. 4, the hue of interlevel is allocated, for example, to 1/4 of the maximum value (1/4 δ_{MAX}) of the strain, not to the intermediate value (δ_{AV}) of the strain. In this manner, the strain between the minimum value δ_{MIN} to 1/4 δ_{MAX} is displayed in colors from blue to green. When this second display mode is compared with the first display mode, while the strain between the minimum value δ_{MIN} and the maximum value δ_{MAX} is displayed to change linearly from blue to red in the first display mode, in the second display mode it is displayed so that the respective hues from the minimum value δ_{MIN} to 1/4 δ_{MAX} changes linearly from blue to green, and from 1/4 δ_{MAX} to the maximum value δ_{MAX} from green to red. In other words, the hue variation is enlarged to display the region with small strain and the hue variation is reduced to display the region with large strain.

In the same manner as the first display mode, it is preferable to display the comparative figure adjacent to color bar 205 also in the second display mode. However, it is preferable to set the reference value of the comparative figure as the maximum value δ_{MAX} while it is difficult to adopt the first display mode setting the reference value of the comparative figure as the average value δ_{AV}. An example displaying the comparative figure using this method is shown in FIG. 3. The comparative figure can be obtained by calculation in CPU 111 based on the above-mentioned relationships, and δ_{MAX} is displayed adjacent to the red color of color bar 205, 5/8 δ_{MAX} adjacent to yellow, 1/4 δ_{MAX} adjacent to green, 1/8 δ_{MAX} adjacent to light blue and δ_{MIN} adjacent to blue.

Next, equipment component and operation for achieving this second display mode will be described. Though this second display mode can be carried out by itself, here the case enabling the strain image and the color bar to switch from the condition being displayed on the monitor by standard display mode to the color bar of the second display mode will be described. In order to change the display mode of the color bar, the color bar switching key is provided to operation panel 112. When an operator manipulates the color bar switching key, the signals are outputted to CPU 111 and the screen of display monitor 108 changes to the color bar switching mode as shown in FIG. 4. This color bar switching mode screen is the original data of the first display mode of the color bar being read out and displayed in graph form. More specifically, the hue which changes red˜green˜blue is allocated to vertical axis and the minimum value (δ_{MIN})˜the maximum value (δ_{MAX}) of the strain is allocated to the horizontal axis of the graph, and two-dimensional coordinate thereof is represented as (strain δ_{X}, hue code C_{Y}) In this case, the hue code C_{Y} is represented as:
C_{Y}=a·δ_{X} (1),
and this function is displayed as line 301 in the graph.

In order for the operator to switch the color bar in relation to this screen, point (δ_{AV}, C_{GREEN}) on line 301 is dragged and moved to (1/4 δ_{MAX}, C_{GREEN}) using an input device such as a mouse. Then CPU 111 changes the line formula represented by the formula (1) to two line formulas of line 302 connecting (δ_{MIN}, C_{BLUE}) and (1/4 δ_{MAX}, C_{GREEN}) and line 303 connecting (1/4 δ_{MAX}, C_{GREEN}) and (δ_{MAX}, C_{RED}). This change of the line formula can be implemented using software for displaying graphs being installed in CPU 111. CPU 111 then recalculates the relationship between the strain on these lines and the hue code and records them into memory of color bar construction unit 110. In addition, while the changing of the line was carried out in the above-mentioned example dragging the point on the line using an input device such as a mouse, the same result can be obtained by inputting the coordinate points from a keyboard.

After the graph representing the relationship between the strain and the hue code is displayed by a polygonal line, when the operator executes the return operation to the screen display mode the hue is assigned to the strain image in the relationship between the strain and the hue code after the above-mentioned change and displayed on the monitor as well as the comparison value is displayed adjacent to color bar 205. In other words, the color bar on the left side of FIG. 4 is displayed along with the strain image.

While the region with small strain is displayed with enlarged hue in the above-mentioned embodiment, it is possible to display the region with large strain also with enlarged hue. In this case, it is possible to do so by, for example, dragging point (δ_{AV}, C_{GREEN}) on the line of formula (1) to (3/4 δ_{MAX}, C_{GREEN}).

It goes without saying that the operator can select at his/her discretion on which point said line 301 should be changed to the polygonal line to carry out the operation.

While an example is cited in the second display mode for representing the relationship between the strain and the hue code in two lines, it also is possible to represent the relationship between the strain and the hue code using more than three lines. The example thereof is shown in FIG. 5. The polygonal line shown in FIG. 5 is formed with lines 401, 402, 403 and 404. In the example of FIG. 5 the hue variation is made great in the regions where the strain is big or small, and the hue variation of the regions where the strain is intermediate level is made small. In this manner, the relationship between the strain and the hue code can be set in a discretional number of lines. As is easy to be assumed, a curve may be used when the number of lines is increased boundlessly.

Furthermore, in accordance with the present invention it is possible to give the same hue code to the region having more than a certain strain value as shown in FIG. 6. In an example shown in FIG. 6, the hue code of red color is given to all the pixels having more than X-times of the average value of the strain. According to this example of display, since the region with large strain is displayed with the same color and only the region with small strain is given with the hue variation, the region where the operator has to carefully observe will be reduced.

In accordance with the present invention it is also possible to provide the hues that are independent such as red, yellow, green gray and black as oppose to provide with the hue code that changes its color from red to blue on a slant, so that the range of the strain having these colors can be covered. This example is shown in FIG. 7. In an example shown in FIG. 7 displays from the minimum value of the strain δ_{MIN} to 1/A δ_{AV} in black, from 1/A δ_{AV} to 1/B δ_{AV} in gray, from 1/B δ_{AV} to C·δ_{AV} including the average value of the strain δ_{AV} in green, from C·δ_{AV} to D·δ_{AV} in yellow and from D·δ_{AV} to the maximum value of the strain δ_{MAX} in red (Note however that 1/A<1/B<C<D here). In this case as shown in FIG. 7, besides δ_{MIN} indicating the minimum value of the strain and δ_{MAX} indicating the maximum value of the strain adjacent to the color bar, 1/A δ_{AV}, 1/B δ_{AV}, C·δ_{AV} and D·δ_{AV} are displayed on the boundaries of the respective hues on the color bar. While the number of hues is five in this example in FIG. 7, this number does not have to be particularly limited.

Even with the above-mentioned display mode of the color bar, it takes a good amount of experience for a doctor to determine where in the color bar the hue of the diseased region of interest in the strain image corresponds. The embodiment to be described next is for saving the time of a doctor in making such determination. FIG. 8 is a diagram showing the embodiment thereof. This embodiment is described in the condition that the screen of FIG. 2˜FIG. 7 is being displayed, and here the condition with FIG. 2 being displayed is described.

In FIG. 8, when a doctor has an interest in affected area 203 in a strain image, they attempt to know the hardness of affected area 203. Then a doctor sets a coordinate point or minute ROI 203A in the affected area using an input device such as a mouse. CPU 111 accesses to the memory and specifies the coordinate point thereof or the hue information assigned to the pixels of minute ROI 203A. CPU 111 then applies the stick-like mark on color bar 205 based on the specified hue information, and displays how much of the strain the pixel has or how much the strain of the pixel is in relation to the reference value of the strain, for example, the average value, to the position adjacent to color bar 205 using the numeric value or mark. The detailed description of the implementation of the above mentioned display mode will be omitted since this display mode can be easily implemented software-wise, because the color bar is already created based on the relationship between the strain and the hue.

While the above-mentioned strain image data is obtained by deformation of organs caused by an examiner such as a doctor pressing ultrasound probe 102 on the body surface of object 101 toward the inside direction of the body, besides displacement of a heart itself, such as heart beats of or the displacement of surrounding tissues caused by heart beats, it is possible by the present invention to create an image indicating elastic modulus of an organ or tissues (elastic image) in place of the strain image, by detecting the pressure caused by pressing the ultrasound probe on the object, and to apply it in case of displaying it synthesizing with a tomogram. Young's modulus, that is one of the indices indicating the elastic modulus, can be calculated as follows:
Ymi,j pressure (stress) i,j/strain value i,j (2).
Here, i,j means coordinates of frame image data, and i,j=1,2,3, . . . .

In order to perform this calculation, the pressure added by the examiner to ultrasound probe 102 is detected by pressure sensor 113. Pressure sensor 113 may be set to detect the pressure directly by setting it on the same surface as the one to which ultrasound probe 102 is applied to object 101. Or it may be mounted in the compression system which is provided with a compression member set on the same surface as the one applying to the body surface of object 101 and the detector system for detecting the compression force being applied to ultrasound probe 102, for performing calculation on the detected compression force dividing by the area of compression member.

Elasticity images are created by constructing images of such applied pressure to the object and Young's modulus Ymi,j being obtained from strain data, and in case of displaying such elasticity images, if the hue is assigned upon display the regions of cancer or tumor can be easily distinguished from normal tissues. Also the relationship between the color bar of the hues and elastic modulus can be applied to the relationship between the color bar of the hues in the above-mentioned strain image display and the strain, which should be easily comprehended by those skilled in the art.

While the present invention has been described above in details, the present invention is not limited to the above-mentioned embodiments, and is possible to apply to all sorts of variations. For example, while an example for carrying out the strain measurement only in ROI is described in the above-mentioned embodiment, it may be carried out in the entire scope of ultrasound measurement. Also, while the average value or the maximum value of the strain measured from body tissues is set as the reference value for representing the quantitation of the strain in the above-mentioned embodiment, the present invention is not limited particularly to such setting. For example, by arranging a material having elasticity between the object and probe and making it possible to measure the strain from the load or pressure applied on the probe, and the strain value of the material thereof may be set as the reference value of the comparative figure.

1. An ultrasonic diagnostic apparatus for creating a color image of strain data by assigning a plurality of hues to the strain data according to the amount of strain of tissues of a living body measured in a scope of ultrasound measurement by transmitting/receiving the ultrasonic waves to/from the inside of the body of an object to be examined and displaying it on a color monitor along with a color bar of the assigned hue information, wherein the ultrasonic diagnostic apparatus comprises means for displaying the comparative information in relation to a measured reference value of the strain.

2. The ultrasonic diagnostic apparatus according to claim 1, wherein the reference value is an average value or a maximum value.

3. The ultrasonic diagnostic apparatus according to claim 1, wherein the comparative information is assigned corresponding to the representative hue of the color bar.

4. The ultrasonic diagnostic apparatus according to claim 1 comprising means for setting the region of interest (ROI) in a scope of ultrasound measurement and means for obtaining an average value or maximum value of the strain from data in the ROI.

5. The ultrasonic diagnostic apparatus according to claim 1, comprising means for displaying the relationship between measured strain data of the tissues in a living body and the assigned hue information on the color monitor.

6. The ultrasonic diagnostic apparatus according to claim 5, wherein the relationship between measured strain data of the tissues in a living body and the assigned hue information is displayed in a two-dimensional graph form with one set as hue code and the other as quantitation.

7. The ultrasonic diagnostic apparatus according to claim 6, having a standard hue conversion display mode for converting the two-dimensional graph indicating the relationship between the measured strain data of the tissues in a living body and the assigned hue information into a straight-line graph.

8. The ultrasonic diagnostic apparatus according to claim 7, comprising means for making the relationship between the measured strain data of the tissues of the living body and the assigned hue information non-linear by rewriting the data of the straight-line graph of the standard hue conversion mode.

9. The ultrasonic diagnostic apparatus according to claim 8, wherein the comparative information being displayed corresponding to the hue of the color bar is changed when the relationship between the measured strain data of the tissues in the living body and the assigned hue information is made non-linear.

10. The ultrasonic diagnostic apparatus according to claim 8, wherein a polygonal line graph is created by dragging the discretional point on the straight-line graph being displayed on a color monitor by the standard hue conversion display mode to a point different from the straight-line graph on a screen, and the relationship between the measured strain data of the tissues in the living body and the assigned hue information is recreated based on the polygonal line graph.

11. The ultrasonic diagnostic apparatus according to claim 9, wherein means for dragging a discretional point on the straight-line graph to a point different from the straight-line graph on a screen is a mouse, joy stick or trackball.

12. The ultrasonic diagnostic apparatus according to claim 8, wherein means for making the relationship between the measured strain data of the tissues in the living body and the assigned hue information a non-linear, is coordinate input means for inputting coordinate points that are in a two-dimensional graph.

13. The ultrasonic diagnostic apparatus according to claim 1 comprising:
means, when the particular positional information of a pixel from the strain image displayed on the color monitor is inputted, for specifying the hue assigned to the measurement data of the pixel position thereof on the color bar, and
means for displaying the comparative information corresponding to the hue specified to the measurement data of the pixel position thereof.

14. (canceled)
